(19) United States
(12) Patent Application Publication
(10) Pub. No.: US 2006/0135887 A1
(43) Pub. Date: Jun. 22, 2006

(54) Uterine sound

(76) Inventors: Russel M. Sampson, Palo Alto, CA (US); Estela H. Hilario, Los Altos, CA (US); J. Brook Burley, Sunnyvale, CA (US)

Correspondence Address: FISH & RICHARDSON P.C., PO BOX 1022, MINNEAPOLIS, MN, 55440-1022, US

(21) Appl. No.: 11/019,500

(22) Filed: Dec. 20, 2004

(51) Int. Cl. A61B 5/103

(52) U.S. Cl. 600/591

(57) ABSTRACT

A uterine sound is configured having an open and a closed position. Under normal operating conditions, the uterine sound is in the closed position for insertion into a uterus to measure the length of the uterus. Under conditions where there is a risk of the uterine sound perforating a uterine wall, the uterine sound switches into an open position. The open position provides an enlarged surface area of a distal end of the uterine sound that is in contact with a uterine wall and resists perforation by the uterine sound of the uterine wall.

### TECHNICAL FIELD

This invention relates to medical devices.

### BACKGROUND

The human uterine cavity is approximately triangular in shape and relatively flat, much like an envelope. The cavity is entered via the endocervical canal. The proximal end of the canal, the external cervical os, opens to the vagina while the distal end, the internal cervical os, opens to the uterine cavity. The tip of the triangular-shaped uterine cavity is located at the internal cervical os, while the base is defined by the openings that lead to the fallopian tubes, the tubal ostia. Sounding the uterus, i.e., determining the length of the uterus, is usually a blind procedure. A physician inserts a uterine sound transcervically and advances the sound until it reaches the top, or fundus of the uterine cavity, i.e., the base of the triangle between the tubal ostia. The length from the interior fundus to the external cervical os can be measured directly using graduations stamped on the shaft of the sound. The physician relies upon tactile feedback to determine when the sound has touched the fundus.

Sounding the uterus can lead to perforation of the uterine wall by the uterine sound, reportedly in as many as 1% to 3% of all intrauterine procedures. The risk of perforation increases when locating the fundus is difficult, especially if the uterine cavity is severely anteverted or retroverted, if intrauterine pathology (e.g., polyps, fibroids) is present, or if the cervix is stenotic. In the latter case, the cervix is difficult to dilate to a diameter sufficient to facilitate easy passage of the sound. A tight cervix can create friction against the sound, requiring more force to be exerted by the physician in order to advance the sound to the fundus. Increased insertion force, uncertain geometry and intracavitary pathology all contribute to increased incidence of perforation.

Conventional uterine sounds are constructed from a malleable metal material, approximately 3.5 mm in diameter with a working length of roughly 25 cm, and have a flattened handle portion the physician can grasp. The uterine sound necessarily is substantially rigid in the axial direction and somewhat flexible out of plane, transverse to its axis, in order to reach the fundus and provide the physician the tactile sensation of touching the fundus. The small diameter and axially rigid construction of the conventional uterine sound makes it relatively easy to perforate the uterine wall. Additionally, the physician cannot easily discern the difference between resistance due to the uterine wall and that due to a tight cervix, further increasing the risk of perforation.

### SUMMARY

In general, in one aspect, the invention features a uterine sounding device including an elongate member having a longitudinal axis, distal and proximal ends. The elongate member, being configured for insertion into a uterus, includes an end cap connected to the distal end of the elongate member. The end cap is in a closed position when the elongate member is inserted into the uterus, subsequently switching into an open position when a force applied to a distal tip of the end cap by the uterus exceeds a threshold force. A surface area of the end cap projected onto a plane substantially perpendicular to the longitudinal axis of the elongate member is enlarged in the open position as compared to in the closed position. The open position resists penetration of the end cap into a wall of the uterus.

Implementations of the invention can include one or more of the following features. The elongate member of the device can be substantially rigid compressively between the distal and proximal ends, and substantially flexible out of the plane of the longitudinal axis. The elongate member of the device can include graduations marked on at least a portion of a length of the elongate member for measuring a length of the uterus.

The end cap of the device can include one or more fin members, where the fin members are positioned along a longitudinal axis of the elongate member when the end cap is in the closed position and deploy laterally from the longitudinal axis when switching into the open position. Each of the one or more fin members of the device can include of a first fin link and a second fin link. Furthermore, when in the open position, the first fin link, the second fin link and a portion of the elongate member can approximately form a triangle. The first fin link of the device can be deployed at substantially 90 degrees to the longitudinal axis of the elongate member and the second fin link can be deployed at substantially 30 degrees to the longitudinal axis of the elongate member. For each fin member of the device, the first fin link and the second fin link can be a unitary element and can be separated by a portion having decreased thickness comprising a living hinge. In one embodiment, the first link can be substantially 0.25 centimeters to 1 centimeter in length and the second fin link can be substantially 0.75 centimeters to 3 centimeters in length. In another embodiment, the first fin link can be substantially 0.7 centimeters in length and the second fin link can be substantially 2.1 centimeters in length.

The device can include a deployment mechanism, configured to switch the end cap from the closed position to the open position upon a force on the distal tip of the end cap exceeding the threshold force. In one implementation, the deployment mechanism can include a spring positioned about the elongate member, wherein the spring is preloaded to exert the threshold force on a first face of a retainer connected to the rod. The threshold force exerted by the spring prevents the rod from translating in a direction away from the end cap, and the retainer includes a second face abutting a housing preventing translation of the rod in a direction toward the end cap. When a force on the distal end of the end cap exceeds the threshold force, the rod translates axially compressing the spring. The distal end of the end cap translates causing the one or more fin members to deploy laterally switching the end cap into the open position. The rod can be attached to a hardstop configured to limit translational movement of the rod in a direction away from the end cap. The device can further include a handle attached to the elongate member substantially near the proximal end of the elongate member, and the deployment mechanism can be housed within the handle.

The distal tip of the end cap of the device can be configured to be atraumatic. For example, the distal tip of the end cap can be a full radius tip, a chamfered tip, or a convex tip.

The device can include a handle attached to the elongate member substantially near the proximal end. The device can further include an indicator configured to provide an indication to a user when the end cap has switched from the closed to the open position. The indication can include at least one or more of the following: a tactile, visual, electric or audible signal. In one implementation, the indicator can be a member that protrudes from the handle when the end cap is in the open position.

In general, in another aspect, the invention features a uterine sounding device including an elongate member having distal and proximal ends. The elongate member is configured for insertion into a uterus and include graduations marked on at least a portion of a length of the elongate member for measuring a length of the uterus. The device further includes an end cap connected to the distal end of the elongate member. The end cap is in a closed position when the elongate member is inserted into the uterus, and switches into an open position when a force applied to a distal tip of the end cap by the uterus exceeds a threshold force. A surface area of the end cap projected onto a plane substantially perpendicular to the longitudinal axis of the elongate member is enlarged in the open position as compared to in the closed position. The open position can resist penetration of the end cap into a wall of the uterus. The device further includes a handle connected to the elongate member substantially near the proximal end. The handle houses a deployment mechanism configured to switch the end cap from the closed position into the open position when the force applied to the distal tip of the end cap exceeds the threshold force.

Implementations of the invention can include one or more of the following features. The end cap can include one or more fin members, where the fin members are positioned along a longitudinal axis of the elongate member when the end cap is in a closed position and deploy laterally from the longitudinal axis when switching into the open position.

The elongate member can include a rod connected to the distal tip of the end cap, wherein the deployment mechanism includes a spring positioned about the elongate member. The spring can be preloaded to exert the threshold force on a first face of a retainer connected to the rod, where the threshold force exerted by the spring prevents the rod from translating in a direction away from the end cap. The retainer includes a second face abutting an inner wall of the handle preventing translation of the rod in a direction toward the end cap. When a force on the distal end of the end cap exceeds the threshold force, the rod translates axially compressing the spring and thereby translating the distal end of the end cap causing the one or more fin members to deploy laterally switching the end cap into the open position.

Aspects of the invention may include one or more of the following advantageous features. When there is no risk of perforating a uterine wall, the uterine sound operates in a closed position, maintains a small insertion profile and is substantially axially rigid. When a force exerted by a uterine wall on the distal tip of the uterine sound exceeds a threshold force, the surface area of the distal tip substantially increases (the open position) and resists perforation of the uterine wall. However, in the closed position, friction on the shaft of the uterine sound from a tight cervix or other conditions does not trigger a switch to the open position, allowing the uterine sound to function without interference in the absence of a risk of perforation.

From the perspective of a user, the uterine sound generally can be used in the same way as a conventional uterine sound, and can therefore easily replace a conventional uterine sound, while providing the benefit of the safer end cap.

The end cap of the uterine sound can include fins having shorter and longer links deployable outwardly, with the shorter links coming to a stop at approximately 90 degrees to the long axis of the elongate member and the longer links stopping at approximately 30 degrees to the long axis. When in the open position, this geometry presents the maximum surface area possible to the uterine wall on the short links, yet creates a rigid, stable triangular configuration that can withstand a substantial load without buckling.

An atraumatic design of the distal tip can prevent scraping the uterine walls and function to resist perforation. Under typical operating conditions, the uterine sound pushes against the distal wall of the uterine cavity in a direction substantially perpendicular to the wall. Intravascular catheters and the like include tips useful in preventing scraping and resisting perforation. However, these are ill suited for use in the current application, as the primary function of the uterine sound is to measure a length and long, flexible tips tend to deflect or buckle, leading to erroneous measurements. The uterine sound includes an atraumatic tip designed to resist perforation in a direction normal to tissue. For example, when the distal tip of the uterine sound is configured as a convex tip, an axial load applied to the end cap generates compressive stress in the central region of the tip. The resulting compressed short column of tissue therein tends not to divide. In order for the tip to perforate, the tissue must be sheared around the outer perimeter, while the central region is compressed which requires much higher axial loads.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A is a top view of a uterine sound in a closed position.

FIG. 1B is a top view of the uterine sound of FIG. 1A in an open position.

FIG. 2 is a top view of the end cap of the uterine sound of FIG. 1A.

FIG. 3 is a top view of the end cap of the uterine sound of FIG. 1B.

FIG. 4 is a cutaway view of a handle of the uterine sound of FIGS. 1A and 1B.

FIG. 5A shows side and end views of a full radius tip of a uterine sound.

FIG. 5B shows side and end views of a chamfered tip of a uterine sound.

FIG. 5C shows side and end views of a convex tip of a uterine sound.

FIG. 6A shows a full radius tip of a uterine sound producing an axial load on the uterine wall.

FIG. 6B shows a chamfered tip of a uterine sound producing an axial load on the uterine wall.

FIG. 6C shows a convex tip of a uterine sound producing an axial load on the uterine wall.

Like reference symbols in the various drawings indicate like elements.

### DETAILED DESCRIPTION

A uterine sound is configured having an open and a closed position. Under normal operating conditions, the uterine sound is in the closed position for insertion into a uterus to measure the length of the uterus. Under conditions where there is a risk of the uterine sound perforating a uterine wall, the uterine sound switches into an open position. The open position provides an enlarged surface area of a distal end of the uterine sound that is in contact with a uterine wall and resists perforation by the uterine sound of the uterine wall.

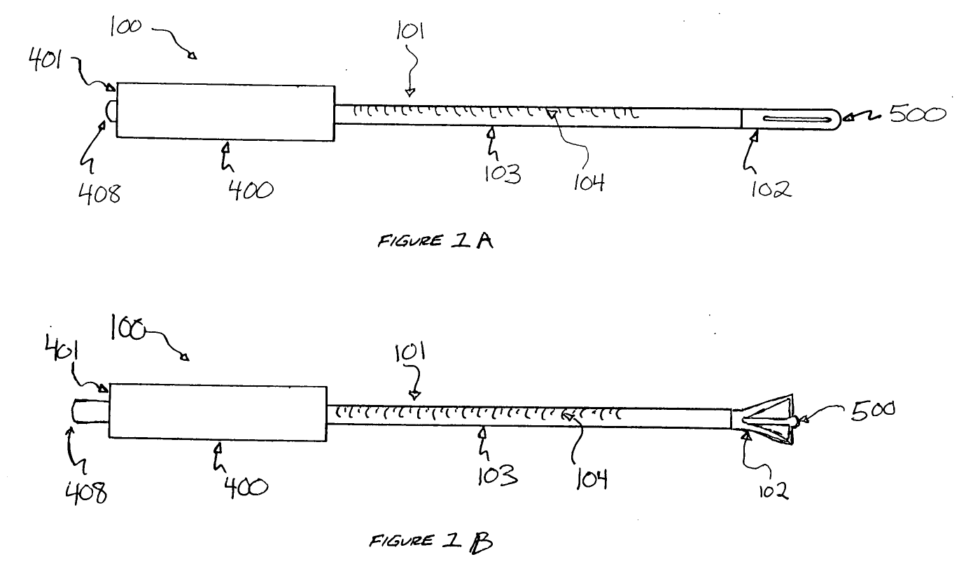

Referring to FIGS. 1A and 1B, one embodiment of a uterine sound 100 is shown. In FIG. 1A the uterine sound 100 is in a closed position, and is configured to facilitate insertion into a uterus. In FIG. 1B the uterine sound 100 is in an open position; the end cap 102 has changed geometry from having a relatively small distal tip to having an enlarged surface area.

In the embodiment depicted, the uterine sound 100 includes an elongate member 101 having distal and proximal ends, where the elongate member 101 is configured for insertion into a uterus. Similar to a conventional uterine sound, the elongate member 101 is generally rigid axially yet flexible and/or malleable non-axially. As such, the elongate member 101 is rigid in the compressive direction with respect to the elongate member's distal and proximal ends, and flexible out of a longitudinal axis of the elongate member 101. The elongate member can be rigid in the compressive direction such that a user is provided a tactile sensation when the fundus of the uterus is engaged. The general configuration of the uterine sound 100 permits the same general procedure for measuring the length of the uterus as a conventional uterine sound.

In one embodiment, the uterine sound 100 is disposable and made of injection molded thermoplastic. The elongate member 101 of the uterine sound 100 can be molded to include a curvature suitable for easing passage of the elongate member 101 through the uterus. The curvature of the elongate member can be configured in any number of shapes and degrees of curvature, including but not limited to for example, the average curvature of the uterus. In another embodiment, the uterine sound 100 is disposable and made of injection molded thermoplastic and a malleable metal. In this embodiment, the elongate member 101 can optionally be molded to include a desired curvature and/or be manipulated by a user to provide a desired curvature.

As shown in FIGS. 1A and 1B, an end cap 102 is connected to the distal end of the elongate member 101. The end cap 102 can be configured in a closed position for when the elongate member 101 is inserted into the uterus and when sounding the uterus under normal conditions (see FIG. 1A). The end cap 102 can further be configured to switch into an open position of enlarged surface area when a force is applied to a distal tip 500 of the end cap 102 by the uterus in excess of a threshold force (see FIG. 1B). That is, the surface area of the uterine sound 100 projected onto a plane substantially perpendicular to a longitudinal axis of the elongate member 101 is enlarged in the open position. In the open position the enlarged geometry of the end cap 102 resists penetration of the uterus by the uterine sound 100. A handle 400 can be connected to the proximal end of the elongate member 101.

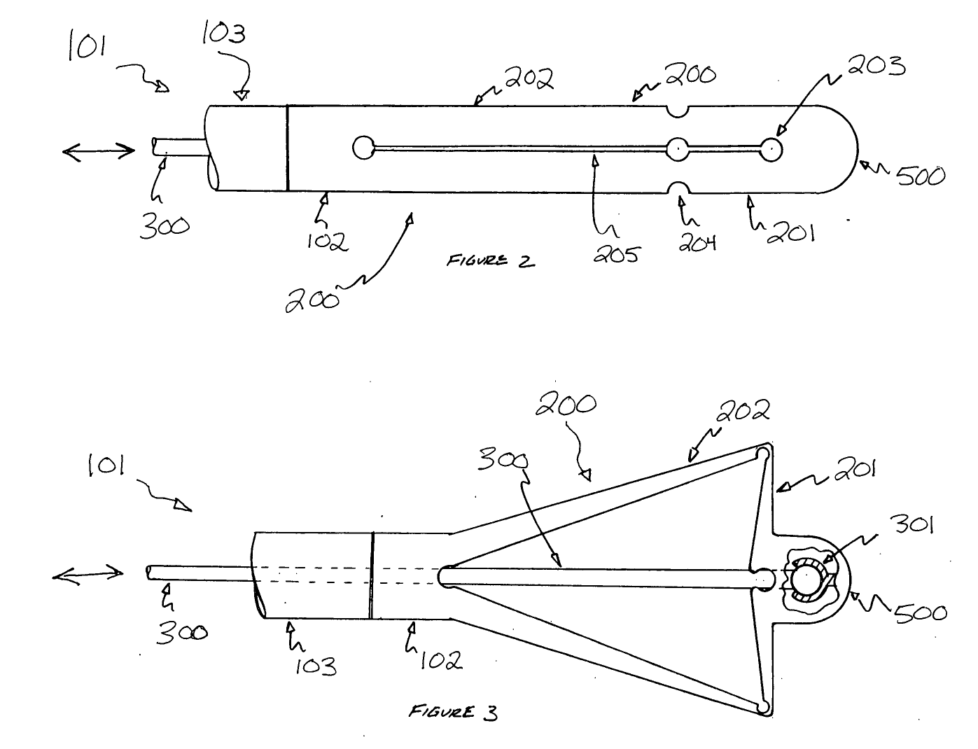

Referring also to FIG. 2, in the embodiment depicted, the elongate member 101 includes a shaft 103 and a rod 300 disposed within the shaft 103. Optionally, the shaft can include graduations 104 (e.g., centimeter graduations) over its length for measuring the length of a uterus. The rod 300 spans the length of the elongate member 101 and is attached to the distal end of the end cap 102. Referring to FIG. 3, in one embodiment the rod 300 is attached to the distal tip 500 of the end cap 102 by a snap fit 301 connection. The snap fit 301 can be in the form of a clevis-type coupling (see FIG. 3) a threaded feature, a pin, a bonding agent or any other suitable means. Where the snap fit 301 is a clevis snap fit, a rotational degree of freedom can be provided between the rod 300 and the distal tip 500 of the end cap 102.

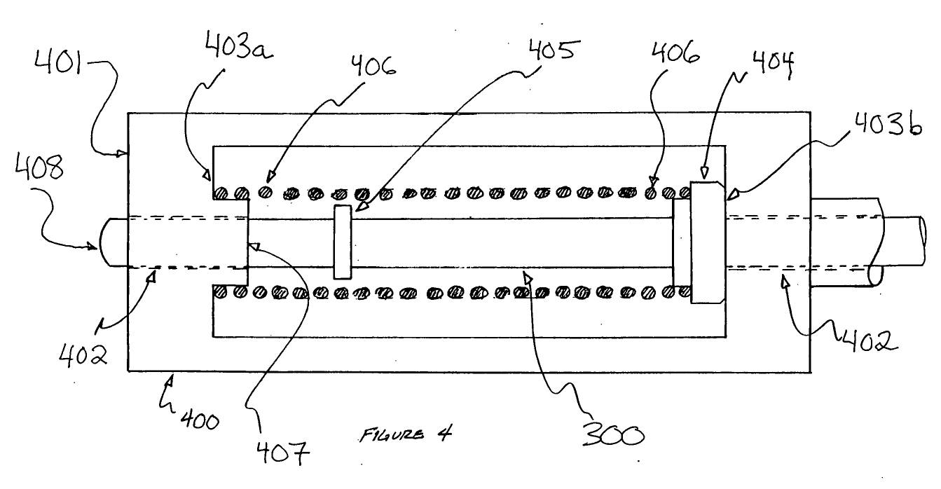

Referring to FIG. 4, the rod 300 can additionally include a hardstop 405 attached to the rod 300 for limiting translational movement of the rod within the handle 400. Also shown in FIG. 4, a retainer 404 can be attached to the rod 300 within the handle 400, which is described further below.

Referring to FIGS. 2 and 3, the end cap 102 can include one or more deployable fins 200 that provide a convertible arrangement for the end cap 102 between a closed position (see FIG. 2) and an open position (see FIG. 3). The open position provides an enlarged surface area at the distal end of the uterine sound 100. Deployment of the end cap 102 to the open position is triggered when a force exceeding a threshold force is exerted on the distal tip 500 of the end cap 102 and transmitted down the shaft 103. That is, when the uterine sound 100 reaches the end of the uterus, or another portion of uterine wall, and a user continues pushing on the proximal end of the uterine sound 100, if the resisting force exerted by the uterine wall on the end cap 102 exceeds the threshold force, then the open position is triggered.

As shown in FIG. 3, in one embodiment, when the open position is triggered, two fins 200 deploy radially outwardly to provide an enlarged surface area. The fins 200 can be formed from shorter links 201 and longer links 202. The length of the shorter links 201 relative to the longer links 202 can follow an approximate 1:3 ratio. Additionally, where the deployed shorter links 201 are substantially perpendicular to the long axis of the uterine sound 100, the longer links 202 are disposed at an angle including but not limited to, for example 25-30°. In one embodiment, the shorter links 201 are approximately 0.25 centimeters to 1 centimeter in length, while the longer links 202 are approximately 0.75 centimeters to 3 centimeters in length. In another embodiment, the shorter links 201 are approximately 0.7 centimeters in length and the longer links 202 are approximately 2.1 centimeters in length. The outward deployment of the shorter links 201 can include rotation of the shorter links 201 through a larger angle than that rotated through by the connected longer links 202. Particularly, the shorter links 201 can be configured to deploy substantially 90 degrees to the long axis of the elongate member 101, while the longer links 202 deploy substantially 30 degrees to the long axis of the elongate member 101 (see FIG. 3). The deployed shorter links 201 and longer links 202 create a substantially rigid, stable triangular configuration capable of withstanding substantial loads without buckling.

The shorter links 201 and longer links 202 of the fins 200 can be injection molded links, pinned rigid links, resilient wire or other suitable formed links. When the end cap fins 200 are injection molded, the end cap 102 can have one or more slots 205 defining fin 200 width and one or more holes 203 in the slot 205. The holes 203 are configured to define shorter link 201 and longer link 202 length, and provide an area of increased bending stress, thereby providing a “living hinge” at the ends of the fins 200. A living hinge can be, for example, a molded thin flexible bridge of material (e.g., polypropylene or polyethylene) that joins two substantially rigid bodies together. Additional one or more holes 204 in the end cap 102 located adjacent to the one or more slots 205, can be configured to enhance the living hinge separating the shorter links 201 and longer links 202.

The uterine sound 100 includes a feature to sense when to switch from a closed to an open position, and a feature to deploy into the open position. In the embodiment shown, a mechanical deployment mechanism both senses when a threshold force is exceeded and automatically deploys the fins 200 into the open position. Referring again to FIGS. 1 and 4, the deployment mechanism can be a mechanical assembly, housed within a handle 400. The handle 400 is attached to the elongate member 101 at or substantially near to the proximal end. Other deployment mechanisms for converting from the closed position to the open position can be used, including electrical means by incorporating a force sensitive resistor (FSR) at the distal tip 500. When the force exerted against the FSR exceeds a threshold value, the resistance of the FSR changes from one state to a different state. A detector located, for instance, in the handle 400 can detect the change and trigger the release of a braking means holding the rod 300 in place, allowing the end cap 102 to deploy. Still another embodiment could employ a pneumatic means, whereby the force applied at the distal tip translates through the rod 300, which could in turn bear on a plunger in a reservoir inside handle 400. When the pressure inside the reservoir reaches the threshold value, a pressure releasing means could trigger the end cap 102 to change to its deployed condition.

An orientation indicator can be provided to indicate to a user the proper orientation of the uterine sound 100 relative to the uterus. For example, where the fins 200 of the uterine sound 100 deploy in a plane, the proper orientation substantially aligns the plane with the plane of the substantially flat uterus to ensure safe deployment of the fins 200. The orientation indicator can be positioned substantially near the proximal end of the uterine sound 100. The orientation indicator can be a marking on the surface, or a tactile indicator at the proximal end of the uterine sound 100. In one embodiment, the proximal end of the handle 400 can include an orientation indicator in the form of a flattened planar side that coincides with the plane of deployment of the fins 200. In one embodiment, the plane of handle 400 itself can indicate the plane of deployment of the fins 200.

In the embodiment shown in FIG. 4, the mechanical assembly included within the handle 400 includes journals 402 for providing a single translational degree of freedom to the rod 300, and a boss 407 for contacting the hardstop 405 of the rod 300, thereby limiting the translational movement of the rod 300. The mechanical assembly further includes a means to govern the threshold force required to trigger conversion to the open position, e.g., to deploy the fins 200. In the embodiment depicted, the means for governing the threshold force include a spring 406, e.g., a compression spring. The spring 406 can be preloaded between the handle wall 403a at the handle's proximal end and the retainer 404 connected to the rod 300 near the handle wall 403b at the handle's distal end. The retainer 404 is constrained by the adjacent handle wall 403b to maintain the spring 406 preload. Alternatively, the governing means can include a pressurized gas in a cylinder formed within handle 400, wherein retainer 404 can be configured as a piston capable of translating through the cylinder.

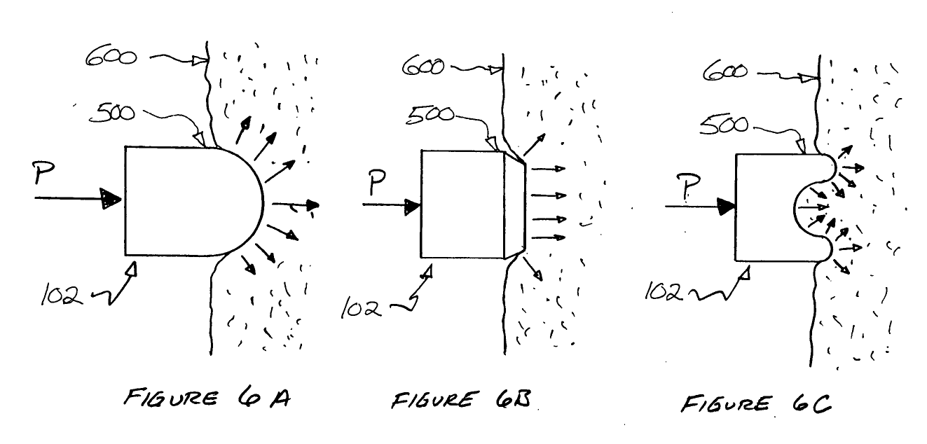

When the uterine sound 100 is inserted into a uterus and the distal tip 500 of the end cap 102 presses against a uterine wall, a resistance force exerted by the uterine wall 600 (see FIG. 6A-C) on the distal tip 500 is transmitted along the rod 300 to the retainer 404. Typically, measurement of the uterus length presents little risk of perforation using the uterine sound 100, since the end of the uterus can be identified by tactile sensation without exceeding the threshold force. Measurement of the uterus length with the uterine sound 100 can include the steps of accessing the cervix with the aid of a speculum, providing traction on the uterus by holding the cervix in position (e.g., using a tenaculum), and inserting the uterine sound 100 through the external cervical os and into the uterus by way of the internal cervical os. Insertion of the uterine sound 100 proceeds until tactile sensation indicates the uterine wall 600 or fundus of the uterus has been reached. Insertion of the uterine sound 100 is ceased, and the point on the uterine sound 100 disposed at the external cervical os is marked. The uterine sound is withdrawn from the cervix, and the length of the uterus based on the marked point on the uterine sound 100 is noted. Graduations included on the elongate member 101 can be used to note the length.

Under certain circumstances, e.g., through inadvertence, accident, anatomical divergence or stenosis of the uterus, the measuring process can result in forces on the uterine wall 600 that could result in perforation of the uterus with the uterine sound 100. For example, in the case of stenosis of the uterus, passing the uterine sound 100 through the cervix may be difficult and lead to an abrupt entry into the uterus. Once a force approaching, but substantially lower than a force capable of perforating the uterine wall 600, i.e., the threshold force, is transmitted to the retainer 404, the force preloaded in the spring 406, i.e., the threshold force, begins to compress the spring 406. As the spring 406 compresses, the retainer 404 moves away from the adjacent handle wall 403b and translates the rod 300 through the journals 402. The rod's translation is limited by the hardstop 405 contacting the boss 407. The translation of the rod 300 relative to the shaft 103 draws the distal tip 500 of the end cap 102 toward the handle 400, thereby deploying the fins 200 (see FIG. 3) and creating the desired enlarged surface area for resisting penetration of the end cap 102 into the uterine wall 600.

After deployment, the fins 200 of the uterine sound 100 can be returned to the undeployed state by e.g., physically pushing the proximal end of the rod 300 to the undeployed position in the elongate member 101, thereby returning the distal tip 500 of the endcap 102 and accordingly the fins 200 to their undeployed positions. Alternatively, in the embodiment depicted, once the force on the distal tip 500 of the end cap 102 is released, i.e., is less than the threshold force, the spring 406 expands and automatically contracts the fins 200. Once returned to the undeployed position, the uterine sound 100 can safely be removed or used again.

Referring again to FIGS. 1 and 4, the uterine sound 100 can optionally include an indicator to indicate to a user of the uterine sound 100 that the threshold force was exceeded and that the uterine sound 100 has converted to the open position. In the embodiment depicted, the indicator is a protrusion 408 from the handle 400 that is continuously connected to the rod 300. When the threshold force of the uterine sound 100 is exceeded, translation of the rod 300 causes the protrusion 408 to further protrude from the handle 400, thereby providing a signal or alert to the user. In other embodiments, the indicator can be both visual and audible and can be a mechanical or an electric device or a combination of the two. For example, where the indicator is the protrusion 408, a colored section (e.g., yellow or red) can be revealed upon exceeding the threshold force when the indicator is caused to protrude further from the handle 400 (not shown).

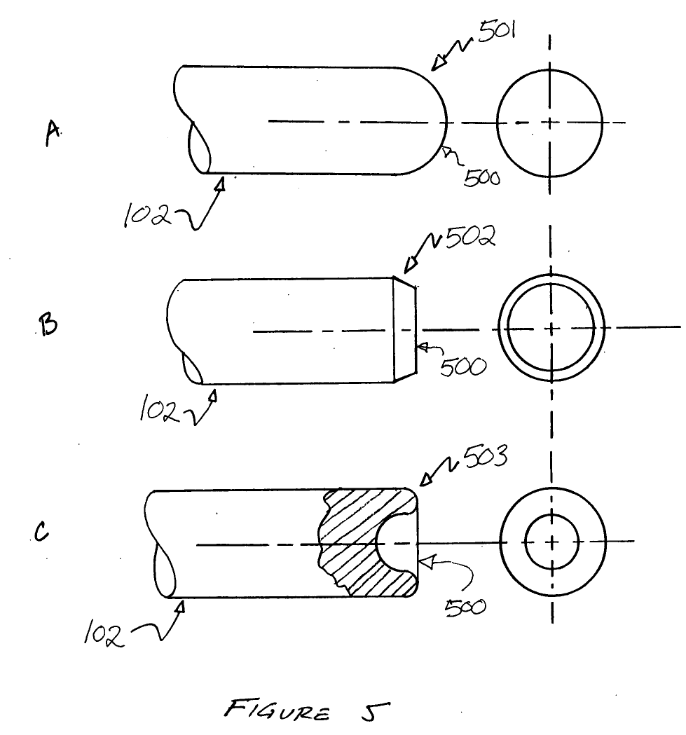

Referring to FIGS. 5A-C and 6A-C, the end cap 102 includes a distal tip 500. The distal tip 500 can include an atraumatic geometry configured to resist perforation of the uterine wall 600 by reducing stress on the uterine wall 600. Examples of atraumatic geometry are shown in FIGS. 5A-C, including a full radius tip 501, a chamfered tip 502 or a convex tip 503 respectively.

As shown in FIGS. 6A-C, different atraumatic distal tip geometries produce different axial loads P on the uterine wall 600. FIG. 6A illustrates the forces on the uterine wall 600 (shown as arrows) by a distal tip 500 configured as a full radius tip 501. FIGS. 6B and 6C similarly illustrate the forces on the uterine wall 600 by distal tips is configured as a chamfered tip 502 and a convex tip 503 respectively. A full radius tip as shown in FIG. 6A, resists scraping the uterine wall 600 during insertion into the uterus, but can tend to divide tissue when an axial load is applied. A chamfered tip 502, as shown in FIG. 6B, resists scraping the uterine wall 600 moderately well and better resists puncturing the wall 600 relative to a full radius tip 501. A chamfered tip 502 tends to create less radial force (indicated by arrows) in tissue, in comparison to a full radius tip 501 as shown in FIGS. 6A and 6B. A convex tip 503 can significantly protect against scraping and puncturing the uterine wall 600 and tends not to divide tissue. As shown in FIG. 6C, although the convex tip 503 does generate some radial forces (indicated by arrows) that develop tensile hoop stress on the outer perimeter, the hoop stress produced in the central region is compressive (indicated by arrows).

A number of embodiments of the invention have been described. Nevertheless, it will be understood that various modifications may be made without departing from the spirit and scope of the invention. Accordingly, other embodiments are within the scope of the following claims.

What is claimed is:

1. A uterine sounding device comprising:
an elongate member having a longitudinal axis, distal and proximal ends, the elongate member configured for insertion into a uterus; and
an end cap connected to the distal end of the elongate member, the end cap being in a closed position when the elongate member is inserted into the uterus and switching into an open position when a force applied to a distal tip of the end cap by the uterus exceeds a threshold force, wherein a surface area of the end cap projected onto a plane substantially perpendicular to the longitudinal axis of the elongate member is enlarged in the open position as compared to in the closed position, and wherein the open position resists penetration of the end cap into a wall of the uterus.

2. The uterine sounding device of claim 1, wherein the elongate member is substantially rigid compressively between the distal and proximal ends, and substantially flexible out of the plane of the longitudinal axis.

3. The uterine sounding device of claim 1, wherein the elongate member further comprises graduations marked on at least a portion of a length of the elongate member for measuring a length of the uterus.

4. The uterine sounding device of claim 1, wherein the end cap includes one or more fin members, where the fin members are positioned along a longitudinal axis of the elongate member when the end cap is in the closed position and deploy laterally from the longitudinal axis when switching into the open position.

5. The uterine sounding device of claim 4, wherein each of the one or more fin members is comprised of a first fin link and a second fin link, and where in the open position the first fin link, the second fin link and a portion of the elongate member approximately form a triangle.

6. The uterine sounding device of claim 5, wherein the first fin link is deployed at approximately 90 degrees to the longitudinal axis of the elongate member and the second fin link is deployed at approximately 30 degrees to the longitudinal axis of the elongate member.

7. The uterine sounding device of claim 5, wherein for each fin member, the first fin link and the second fin link are a unitary element and are separated by a portion having decreased thickness comprising a living hinge.

8. The uterine sounding device of claim 5, wherein for each fin member the first link is approximately 0.25 centimeters to 1 centimeter in length and the second fin link is approximately 0.75 centimeters to 3 centimeters in length.

9. The uterine sounding device of claim 5, wherein for each fin member the first fin link is approximately 0.7 centimeters in length and the second fin link is approximately 2.1 centimeters in length.

10. The uterine sounding device of claim 1, further comprising:
a deployment mechanism, configured to switch the end cap from the closed position to the open position upon a force on the distal tip of the end cap exceeding the threshold force.

11. The uterine sounding device of claim 10, wherein:
the end cap includes one or more fin members, where the fin members are positioned along a longitudinal axis of the elongate member when the end cap is in a closed position and deploy laterally from the longitudinal axis when switching into the open position;
the elongate member includes a rod connected to the distal tip of the end cap; and
the deployment mechanism includes a spring positioned about the elongate member, the spring preloaded to exert the threshold force on a first face of a retainer connected to the rod, where the threshold force exerted by the spring prevents the rod from translating in a direction away from the end cap and where the retainer includes a second face abutting a housing preventing translation of the rod in a direction toward the end cap,
wherein when a force on the distal end of the end cap exceeds the threshold force, the rod translates axially compressing the spring and thereby translating the distal end of the end cap causing the one or more fin members to deploy laterally switching the end cap into the open position.

12. The uterine sounding device of claim 11, wherein the rod is attached to a hardstop configured to limit translational movement of the rod in a direction away from the end cap.

13. The uterine sounding device of claim 11, further comprising:
a handle attached to the elongate member substantially near the proximal end of the elongate member, and wherein the deployment mechanism is housed within the handle.

14. The uterine sounding device of claim 1, wherein the distal tip of the end cap is configured to be atraumatic.

15. The uterine sounding device of claim 1, wherein the distal tip of the end cap is a full radius tip.

16. The uterine sounding device of claim 1, wherein the distal tip of the end cap is a chamfered tip.

17. The uterine sounding device of claim 1, wherein the distal tip of the end cap is a convex tip.

18. The uterine sounding device of claim 1, further comprising:
a handle attached to the elongate member substantially near the proximal end.

19. The uterine sounding device of claim 1, further comprising;
an indicator configured to provide an indication to a user when the end cap has switched from the closed to the open position.

20. The uterine sounding device of claim 19, wherein the indication includes at least one or more of the following: a tactile, visual, electric or audible signal.

21. The uterine sounding device of claim 19, further comprising:
a handle attached to the elongate member substantially near the proximal end; and
wherein the indicator comprises a member that protrudes from the handle when the end cap is in the open position.

22. A uterine sounding device comprising:
an elongate member having distal and proximal ends, the elongate member configured for insertion into a uterus and including graduations marked on at least a portion of a length of the elongate member for measuring a length of the uterus;
an end cap connected to the distal end of the elongate member, the end cap being in a closed position when the elongate member is inserted into the uterus and switching into an open position when a force applied to a distal tip of the end cap by the uterus exceeds a threshold force, wherein a surface area of the end cap projected onto a plane substantially perpendicular to the longitudinal axis of the elongate member is enlarged in the open position as compared to in the closed position, and wherein the open position resists penetration of the end cap into a wall of the uterus; and
a handle connected to the elongate member substantially near the proximal end, the handle housing a deployment mechanism configured to switch the end cap from the closed position into the open position when the force applied to the distal tip of the end cap exceeds the threshold force.

23. The uterine sounding device of claim 22, wherein the end cap includes one or more fin members, where the fin members are positioned along a longitudinal axis of the elongate member when the end cap is in a closed position and deploy laterally from the longitudinal axis when switching into the open position.

24. The uterine sounding device of claim 23, wherein:
the elongate member includes a rod connected to the distal tip of the end cap; and
the deployment mechanism includes a spring positioned about the elongate member, the spring preloaded to exert the threshold force on a first face of a retainer connected to the rod, where the threshold force exerted by the spring prevents the rod from translating in a direction away from the end cap and where the retainer includes a second face abutting an inner wall of the handle preventing translation of the rod in a direction toward the end cap,
wherein when a force on the distal end of the end cap exceeds the threshold force, the rod translates axially compressing the spring and thereby translating the distal end of the end cap causing the one or more fin members to deploy laterally switching the end cap into the open position.
